(19) United States
(12) Patent Application Publication
(10) Pub. No.: US 2024/0189093 A1
(43) Pub. Date: Jun. 13, 2024

(54) FENESTRATED STENT GRAFT DEVICES AND METHODS OF DEPLOYING FENESTRATED STENT GRAFT DEVICES

(71) Applicant: Mayo Foundation for Medical Education and Research, Rochester, MN (US)

(72) Inventor: Tiziano Tallarita, Altoona, WI (US)

(21) Appl. No.: 18/286,669

(22) PCT Filed: May 17, 2022
(86) PCT No.: PCT/US2022/029589

§ 371 Date: Oct. 12, 2023

Related U.S. Application Data
(60) Provisional application No. 63/189,961, filed on May 18, 2021.

(51) Int. Cl. A61F 2/07; A61F 2/954; A61F 2/958; A61F 2/966

(52) U.S. Cl. CPC A61F 2/07; A61F 2/954; A61F 2/958; A61F 2/9662

(57) ABSTRACT

This document describes devices for treating atherosclerosis and methods for their use. For example, this document describes stent graft devices that include a fenestration or end scallop that is advantageous for use at a bifurcation of a vessel. Specifically, a medical device with a catheter comprising an elongate shaft with a proximal balloon member at a distal end, a distal balloon at the distal end spaced apart from the proximal balloon and a lateral opening in the shaft between the proximal and distal balloon members. The device also comprises a stent graft constructed of a wire framework and cover material, and a fenestration defined through the framework. The stent graft is disposed on the proximal and distal balloon members.

### CROSS-REFERENCE TO RELATED APPLICATIONS

This application claims the benefit of U.S. Provisional Application Ser. No. 63/189,961, filed May 18, 2021. The disclosure of the prior application is considered part of (and is incorporated by reference in) the disclosure of this application.

### BACKGROUND

### 1. Technical Field

This document relates to devices for treating atherosclerosis and methods for their use. For example, this document relates to stent graft devices that include a fenestration or end scallop.

### 2. Background Information

Atherosclerosis is thickening or hardening of the arteries caused by a buildup of plaque in the inner lining of an artery. Risk factors may include high cholesterol and triglyceride levels, high blood pressure, smoking, diabetes, obesity, physical activity, and eating saturated fats.

Sometimes plaque can be present where a branch vessel extends from an artery. Such plaque can be difficult to treat by placing a stent because the resulting position of the stent may tend to cause a partial obstruction of the branch vessel. Accordingly, special stents are beneficial for such situations.

### SUMMARY

This document describes devices for treating atherosclerosis and methods for their use. For example, this document describes stent graft devices that include a fenestration or end scallop.

In one aspect, this disclosure is directed to a stent device that includes a wire framework and a covering material disposed on and covering the wire framework. The stent device defines a fenestration through the wire framework and the covering material.

Such a stent device may optionally include one or more of the following features. The stent device may also include a first release mechanism and a second release mechanism. The first release mechanism may be arranged to remove a localized radial constraint from the stent device at the fenestration. The second release mechanism may be arranged to remove a radial constraint from all portions of the stent device except at the fenestration.

In another aspect, this disclosure is directed to a method of deploying a stent device in an artery with a branch vessel. The method includes positioning the stent device as described herein in the artery at a location of the branch vessel. The method further includes actuating the first release mechanism and, after actuating the first release mechanism, actuating the second release mechanism.

Such a method may optionally include one or more of the following features. The method may also include, after actuating the first release mechanism but prior to actuating the second release mechanism, confirming that the fenestration is in alignment with the branch vessel. The confirming that the fenestration is in alignment with the branch vessel may be performed by injecting contrast agent into the stent device such that the contrast agent passes through the fenestration and into the branch vessel. The confirming that the fenestration is in alignment with the branch vessel may be performed by advancing a guidewire into the stent device, out of the fenestration, and into the branch vessel.

In another aspect, this disclosure is directed to a medical device system that includes a catheter and a stent graft device that is releasably coupled to a distal end portion of the catheter. The catheter includes: (i) an elongate shaft that defines a longitudinal axis, (ii) a proximal balloon member attached to a distal end portion of the shaft, and (iii) a distal balloon member attached to the distal end portion of the shaft. The proximal and distal balloon members are spaced apart from each other. The shaft defines a lateral opening located in the space between the proximal and distal balloon members. The opening is in fluid communication with a first lumen that is defined by the shaft. The stent graft device is constructed of a wire framework and a covering material disposed on and covering the wire framework. The stent graft device includes a proximal portion, a distal portion, and a mid-body portion disposed between the proximal and distal portions. The mid-body portion of the stent graft device defines a fenestration through the wire framework and the covering material. The proximal portion of the stent graft device is disposed on the proximal balloon member. The distal portion of the stent graft device is disposed on the distal balloon member. The fenestration of the stent graft device is radially and longitudinally aligned with the opening defined by the shaft of the catheter.

Such a medical device system may optionally include one or more of the following features. The system may also include a first release mechanism that, when activated, removes a localized radial constraint of the mid-body portion of the stent graft device to thereby allow the mid-body portion of the stent graft device to radially self-expand. The proximal end portion of the stent graft device may be configured to expand in response to inflation of the proximal balloon member. The distal end portion of the stent graft device may be configured to expand in response to inflation of the distal balloon member. The system may also include a wire that is slidably disposable in the first lumen and extendable through the opening defined by the shaft and the fenestration defined by the mid-body portion of the stent graft device. The wire framework may include Nitinol struts in the mid-body portion and stainless steel struts in the proximal and distal portions.

In another aspect, this disclosure is directed to a method of deploying a stent graft device in an artery with a branch vessel. The method includes: (i) advancing any of the systems described herein in the artery until the distal end portion of the catheter and the stent graft device are positioned at a location of the branch vessel: (ii) actuating a first release mechanism to allow the mid-body portion of the stent graft device to radially self-expand: (iii) after actuating the first release mechanism, confirming that the fenestration defined by the mid-body portion of the stent graft device is in alignment with the branch vessel: and (iv) after confirming that the fenestration defined by the mid-body portion of the stent graft device is in alignment with the branch vessel, inflating the proximal and distal balloon members to cause the proximal and distal end portions of the stent graft device to radially expand into contact with the artery both proximally and distally of the branch vessel.

Such a method of deploying a stent graft device in an artery with a branch vessel may optionally include one or more of the following features. The confirming that the fenestration defined by the mid-body portion of the stent graft device is in alignment with the branch vessel may include: (a) injecting contrast agent into the first lumen defined by the shaft: and (b) visually confirming under fluoroscopy that the contrast agent passes: (i) out of the opening defined by the shaft, (ii) through the fenestration defined by the mid-body portion of the stent graft device, and (iii) into the branch vessel. The confirming that the fenestration defined by the mid-body portion of the stent graft device is in alignment with the branch vessel may include: (a) advancing a wire into the first lumen defined by the shaft: and (b) visually confirming under fluoroscopy that the wire passes: (i) out of the opening defined by the shaft, (ii) through the fenestration defined by the mid-body portion of the stent graft device, and (iii) into the branch vessel.

Particular embodiments of the subject matter described in this document can be implemented to realize one or more of the following advantages. In some embodiments, medical conditions such as atherosclerosis and others can be treated using the devices and methods provided herein. In some embodiments, various vascular conditions can be treated in a minimally invasive fashion using the devices and methods provided herein. Such minimally invasive techniques can reduce recovery times, patient discomfort, and treatment costs.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description herein. Other features, objects, and advantages of the invention will be apparent from the description and drawings, and from the claims.

### BRIEF DESCRIPTION OF THE DRAWINGS

FIG. 1 depicts a bifurcated artery with the presence of plaque near the bifurcation.

FIG. 2 depicts the placement of a stent to cover some of the plaque while taking care to leave the branch vessel unobstructed.

FIG. 3 depicts the placement of a stent to cover all of the plaque while obstructing the branch vessel.

FIG. 4 depicts the placement of a Bivio stent as described herein (with its fenestration) to cover the plaque without jailing (obstructing) the branch vessel.

FIGS. 5-7 depict an example two-stage deployment technique for the Bivio stents as described herein.

FIGS. 8-10 depict an example two-stage deployment and fenestration location confirmation technique for the Bivio stents as described herein.

FIGS. 11 and 12 provide further information about the Bivio stents and deployment techniques as described herein.

FIG. 13 depicts an example cylindrical Bivio stent as described herein.

FIG. 14 depicts an example diametrically tapered Bivio stent as described herein.

FIG. 15 describes another deployment technique for the Bivio stents described herein.

FIGS. 16A-16F shows examples of Bivio stents that include fenestrations, and other examples that include an end scallop.

FIGS. 17A-17D show another example of a Bivio stent in various stages of deployment.

Like reference numbers represent corresponding parts throughout.

### DETAILED DESCRIPTION OF EXAMPLE EMBODIMENTS

This document describes devices for treating atherosclerosis and methods for their use. For example, this document describes stent graft devices that include a fenestration or end scallop.

The stents describe herein can also be referred to as “Bivio” stents. The Bivio stents are covered stents or stent grafts (e.g., including a wire framework covered with a fabric such as ePTFE) with one fenestration (a “window” or “opening” through the stent framework and fabric) in the middle of the stent, or a scallop (the absence of the fabric with a U shape) at an end of the stent. The Bivio stents described herein are well suited for use where an artery bifurcates, e.g., an iliac, femoral bifurcation, popliteal bifurcation, or anywhere a large collateral must be preserved after stenting. In some embodiments, the Bivio stents are self-expandable stents.

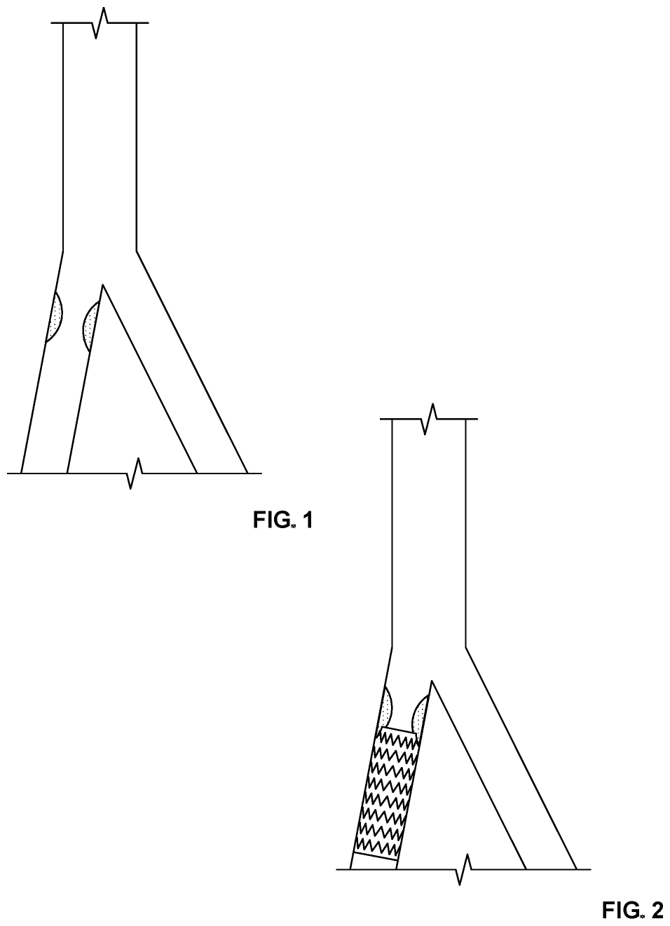

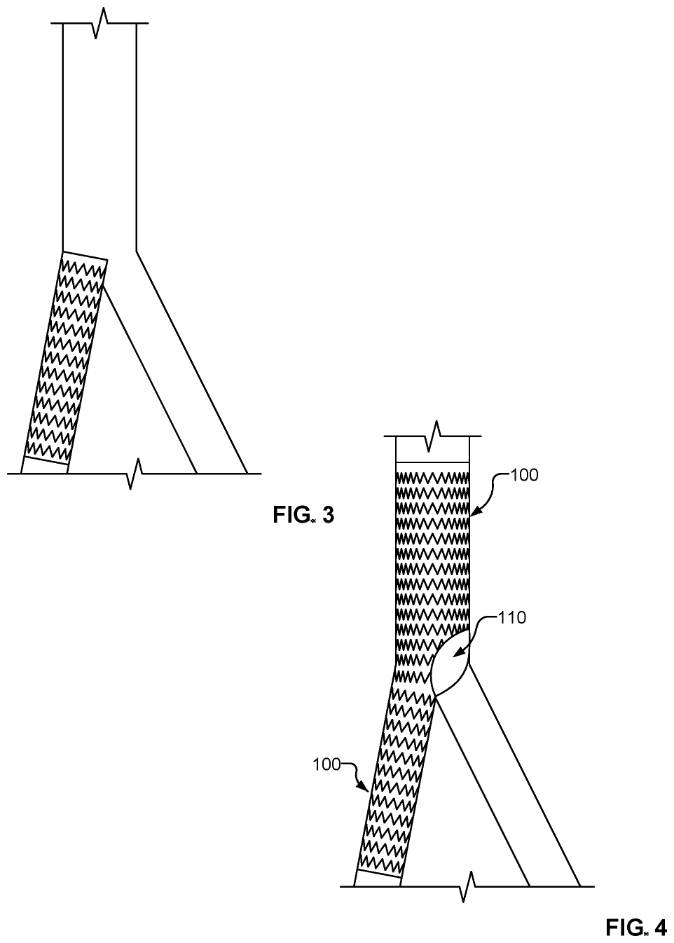

The name “Bivio” means in Italian “crossroads,” which is where the stent should be used “in the arterial tree crossroads.” This idea was born from the frequent need for such a stent in actual practice. In fact, as illustrated in FIG. 1, one of the most common location of atherosclerotic plaque is right at the bifurcation or crossroads of blood vessels, secondary to the turbulence of the flow. Oftentimes practitioners cannot be aggressive in treating the entire stenosis because of a fear of covering the bifurcation (as depicted by FIGS. 2 and 3). However, as depicted in FIG. 4, the Bivio stent 100 with its fenestration 110 can be used to cover the plaque without jailing the branch vessel.

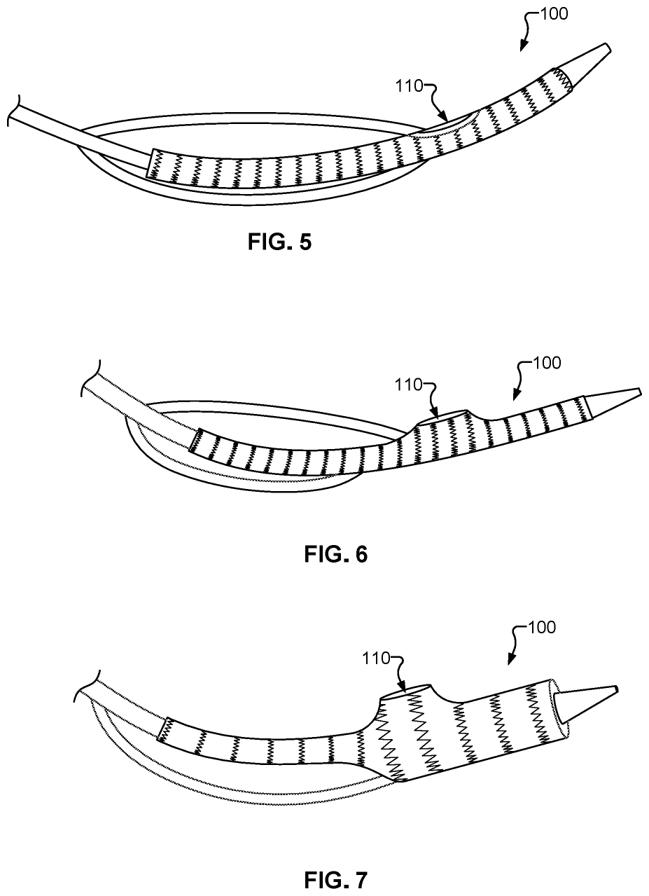

As depicted in the series of FIGS. 5-7, an important feature of the Bivio stent is the two-stage release mechanisms that allows the practitioner to “open the window;” i.e., the mid-body portion of the stent 100 that includes the fenestration 110 (while the rest of the stent 100 is still radially constrained). FIGS. 5 and 6 depict the radial constraint release of the mid-body portion of the stent 100 that includes the fenestration 110 using a first release mechanism (e.g., a first removable wrap or sheath that radially constrains a portion of the stent 100). FIG. 7 depicts the beginning of the use of a second release mechanism (e.g., a second removable wrap or sheath) that removes the radial constraint of the remainder of the stent 100 (i.e., the portions of the stent 100 that are distal and proximal of the mid-body portion).

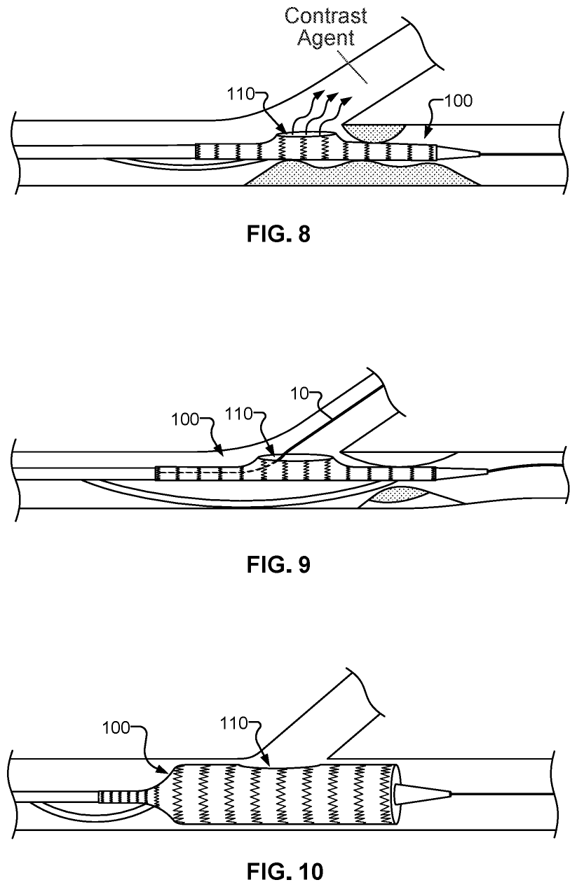

As depicted in the series of FIGS. 8-10, the deployment of the stent 100 actually takes place in situ at the location of an artery bifurcation. FIGS. 8 and 9 depict the radial constraint release of the mid-body portion of the stent 100 that includes the fenestration 110 using the first release mechanism (e.g., removable wrap or sheath). After that, there is a need to confirm that the fenestration 110 is properly positioned and oriented relative to the branch vessel. Accordingly, as depicted in FIG. 8, in some embodiments contrast agent can be inject through the fenestration 110 and, under fluoroscopy, the visualization of the passage of the contrast from the fenestration 110 can be used to align the fenestration 110 to the origin of the branch vessel in order to align the fenestration 110 and the branch vessel with maximal precision. Or, as depicted in FIG. 9, a guidewire 10 can be passed through the fenestration 110 and into the branch vessel to confirm the proper position and orientation of the fenestration 110 using fluoroscopy.

After the alignment of the fenestration with the branch vessel, as depicted in FIG. 10, the distal portion followed by the proximal portion of the stent 100 will be released (allowed to radially expand) in a staged fashion for excellent control. As when we build a structure, this type of stent 100 can in some cases function like a “connector” for side branches of the tree.

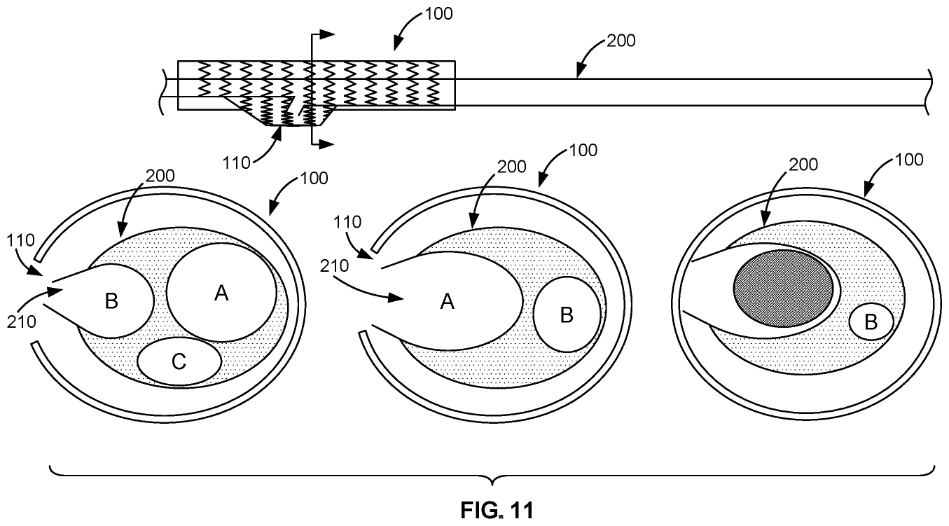

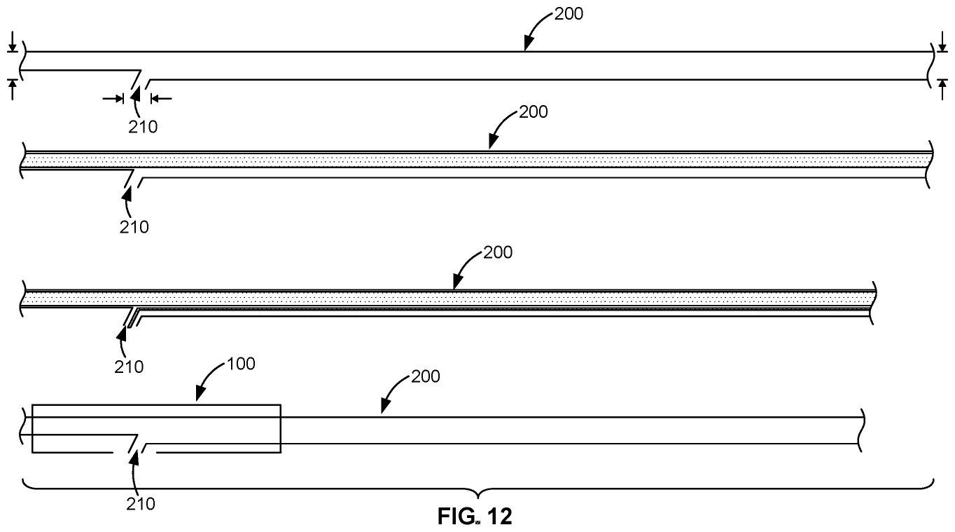

FIGS. 11 and 12 show that the stent 100 can be deployed using various types of a catheter 200. That is, three different cross-sections of a catheter 200 are shown. The catheter 200 can define multiple lumens. A lumen of the catheter 200 can be used for advancing the catheter 200 over a guidewire. Another lumen of the catheter 200 can be used for the delivery of contrast agent or a guidewire as described above in reference to FIGS. 8 and 9. Such a lumen can terminate at a lateral opening 210 extending through a sidewall of the catheter 200 that is aligned with the fenestration 110 of the stent 100. In some embodiments, another lumen can be used to contain the release mechanisms (or to provide a lumen for an inflation medium to be delivered to a balloon device, as described further below).

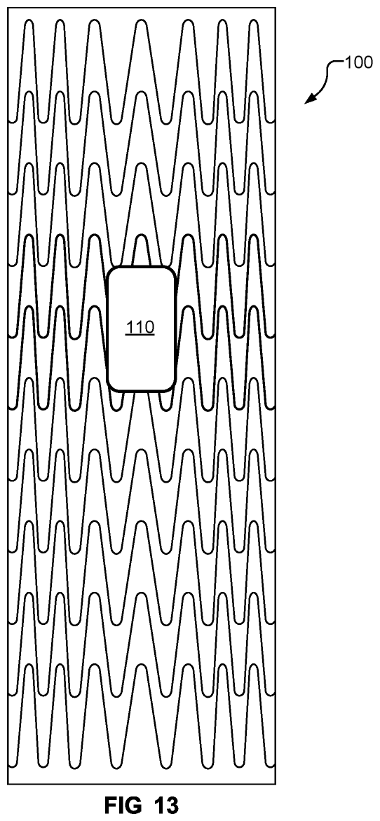

In some embodiments, as depicted in FIG. 13, the Bivio stents 100 can be cylindrical in their radially expanded configuration.

Alternatively, as depicted in FIG. 14, in some embodiments the stent 100′ can be tapered in diameter for better fit of the vessel (e.g., a diameter of 10 mm on top in the main vessel and a diameter of 8 mm bottom where the vessel splits and has smaller diameter).

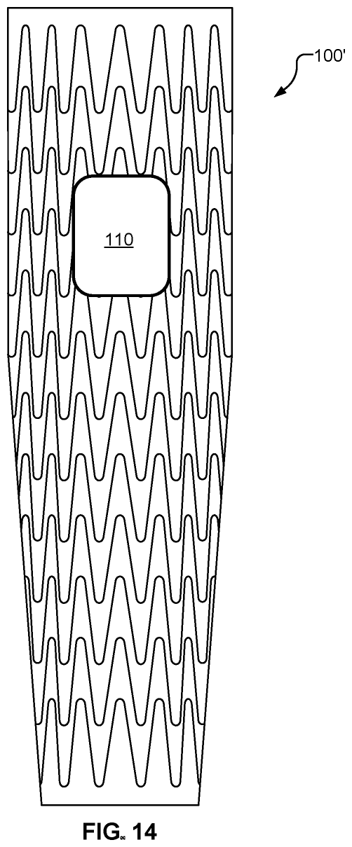

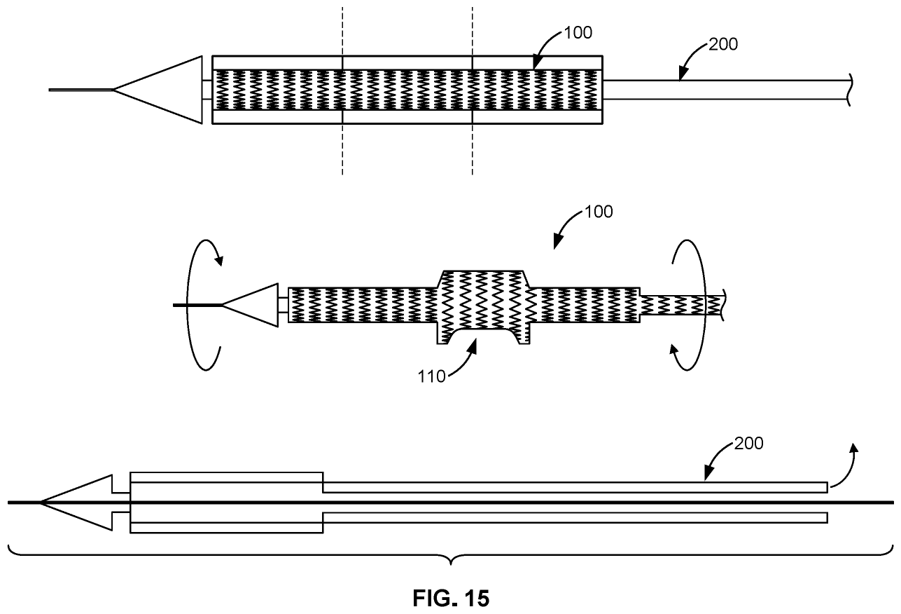

FIG. 15 describes another deployment technique for the Bivio stents 100 described herein. The deployment technique includes the use of multiple sheaths. A first sheath can be pulled back to allow the mid-body portion to radially expand. Thereafter, one or more sheaths can be pulled back to allow the proximal and distal portions of the stent 100 to expand. When the mid-body portion alone is expanded, the stent 100 can be rotated about its longitudinal axis to align the fenestration 110 in the desired radial location (e.g., to align with a vessel).

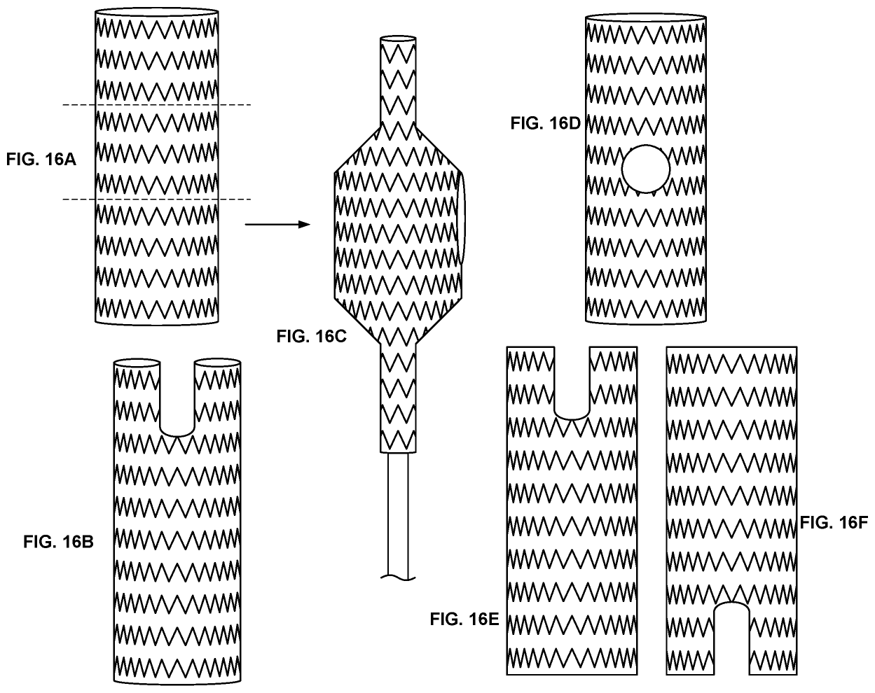

FIGS. 16A-16F shows additional examples of Bivio stents that include fenestrations, a bifurcation, and/or other examples that include one or more end scallops.

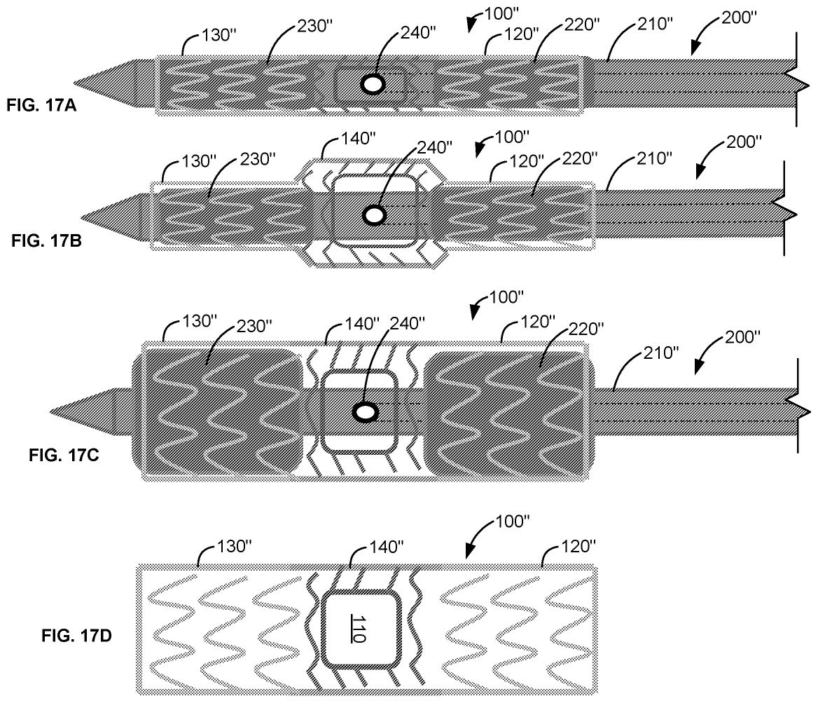

FIGS. 17A-17D show another example of a Bivio stent 100″ that is in various stages of deployment (e.g., FIG. 17A shows the stent 100″ in a fully radially constrained delivery configuration and FIG. 17D shows the stent 100″ in fully deployed configuration). The stent 100″ includes a fabric covering on a wire framework, except for the opening of the fenestration 110.

In some embodiments, the stent 100″ is deployed using a catheter 200″. The catheter 200″ includes: (i) an elongate shaft 210″ that defines a longitudinal axis, (ii) a proximal balloon member 220″ attached to a distal end portion of the shaft 210″, and (iii) a distal balloon member 230″ attached to the distal end portion of the shaft 210″. The proximal balloon member 220″ and distal balloon member 230″ are spaced apart from each other. The shaft 210″ defines a lateral opening 240″ located in the space between the proximal balloon member 220″ and distal balloon member 230″. The opening 240″ is in fluid communication with a first lumen (not visible) that is defined by the shaft 210″. Contrast agent and/or a guidewire can be advanced through the first lumen and the opening 240″ to confirm whether the fenestration 110 is aligned with a branch vessel (as described above in reference to FIGS. 8 and 9). The shaft 210″ of the catheter 200″ can also define a second lumen that can be used to advance the catheter 200″ over a guidewire.

The stent 100″ is releasably coupled to the distal end portion of the catheter 200″ when the stent 100″ and the catheter 200″ system is in the delivery and partially deployed configurations (FIGS. 17A-17C). The stent 100″ is constructed of a wire framework and a covering material disposed on and covering the wire framework. Hence, the stent 100″ can be referred to as a stent graft device. The stent 100″ includes a proximal portion 120″, a distal portion 130″, and a mid-body portion 140″ disposed between the proximal portion 120″ and the distal portion 130″. The mid-body portion 140″ of the stent 100″ defines a fenestration 110 through the wire framework and the covering material.

When the stent 100″ and the catheter 200″ system is in the delivery and partially deployed configurations, the proximal portion 120″ of the stent 100″ is disposed on the proximal balloon member 220″: the distal portion 130″ of the stent 100″ is disposed on the distal balloon member 230″: and the fenestration 110 of the stent 100″ is radially and longitudinally aligned with the lateral opening 240″ defined by the shaft 210″ of the catheter 200″.

The stent 100″ includes a first release mechanism that, when activated, removes a localized radial constraint of the mid-body portion 140″ of the stent 100″. This allows the mid-body portion 140″ to radially self-expand as depicted in FIG. 17B. The proximal end portion 120″ of the stent 100″ is configured to expand in response to inflation of the proximal balloon member 220″. The distal end portion 130″ of the stent 100″ is configured to expand in response to inflation of the distal balloon member 230″. Accordingly, the mid-body portion 140″ is self-expanding, while the proximal end portion 120″ and the distal end portion 130″ of the stent 100″ are balloon expandable. In some embodiments, the mid-body portion 140″ is constructed of shape-memory Nitinol, while the proximal end portion 120″ and the distal end portion 130″ of the stent 100″ are constructed of stainless steel.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described herein should not be understood as requiring such separation in all embodiments, and it should be understood that the described program components and systems can generally be integrated together in a single product or packaged into multiple products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

1. A medical device system comprising:
a catheter comprising: (i) an elongate shaft that defines a longitudinal axis, (ii) a proximal balloon member attached to a distal end portion of the shaft, and (iii) a distal balloon member attached to the distal end portion of the shaft, wherein the proximal and distal balloon members are spaced apart from each other, wherein the shaft defines a lateral opening located in the space between the proximal and distal balloon members, and wherein the opening is in fluid communication with a first lumen that is defined by the shaft; and
a stent graft device releasably coupled to the distal end portion of the catheter, the stent graft device constructed of a wire framework and a covering material disposed on and covering the wire framework, wherein the stent graft device includes a proximal portion, a distal portion, and a mid-body portion disposed between the proximal and distal portions,
wherein the mid-body portion of the stent graft device defines a fenestration through the wire framework and the covering material, and
wherein the proximal portion of the stent graft device is disposed on the proximal balloon member, the distal portion of the of the stent graft device is disposed on the distal balloon member, and the fenestration of the stent graft device is radially and longitudinally aligned with the opening defined by the shaft of the catheter.

2. The system of claim 1, further comprising a first release mechanism that, when activated, removes a localized radial constraint of the mid-body portion of the stent graft device to thereby allow the mid-body portion of the stent graft device to radially self-expand, wherein the proximal end portion of the stent graft device is configured to expand in response to inflation of the proximal balloon member, and wherein the distal end portion of the stent graft device is configured to expand in response to inflation of the distal balloon member.

3. The system of claim 1, further comprising a wire that is slidably disposable in the first lumen and extendable through the opening defined by the shaft and the fenestration defined by the mid-body portion of the stent graft device.

4. The system of claim 1, wherein the wire framework comprises Nitinol struts in the mid-body portion and stainless steel struts in the proximal and distal portions.

5. A method of deploying a stent graft device in an artery with a branch vessel, the method comprising:
advancing the system of any one of claims 1 through 4 in the artery until the distal end portion of the catheter and the stent graft device are positioned at a location of the branch vessel;
actuating a first release mechanism to allow the mid-body portion of the stent graft device to radially self-expand;
after actuating the first release mechanism, confirming that the fenestration defined by the mid-body portion of the stent graft device is in alignment with the branch vessel; and
after confirming that the fenestration defined by the mid-body portion of the stent graft device is in alignment with the branch vessel, inflating the proximal and distal balloon members to cause the proximal and distal end portions of the stent graft device to radially expand into contact with the artery both proximally and distally of the branch vessel.

6. The method of claim 5, wherein the confirming that the fenestration defined by the mid-body portion of the stent graft device is in alignment with the branch vessel comprises:
injecting contrast agent into the first lumen defined by the shaft; and
visually confirming under fluoroscopy that the contrast agent passes: (i) out of the opening defined by the shaft, (ii) through the fenestration defined by the mid-body portion of the stent graft device, and (iii) into the branch vessel.

7. The method of claim 5, wherein the confirming that the fenestration defined by the mid-body portion of the stent graft device is in alignment with the branch vessel comprises:
advancing a wire into the first lumen defined by the shaft; and
visually confirming under fluoroscopy that the wire passes: (i) out of the opening defined by the shaft, (ii) through the fenestration defined by the mid-body portion of the stent graft device, and (iii) into the branch vessel.
